(19) United States
(12) Patent Application Publication
(10) Pub. No.: US 2008/0063731 A1
(43) Pub. Date: Mar. 13, 2008

(54) Mineral infused polyol and method to improve metabolism of individual

(76) Inventors: Stephen B. Roman, Phoenix, AZ (US); Gary S. Kehoe, Phoenix, AZ (US); Cecile E. Kehoe, Phoenix, AZ (US)

Correspondence Address: TOD R NISSLE, PO BOX 55630, PHOENIX, AZ, 85078, US

(21) Appl. No.: 11/900,783

(22) Filed: Sep. 12, 2007

Related U.S. Application Data
(60) Provisional application No. 60/844,004, filed on Sep. 12, 2006.

(51) Int. Cl. A61K 33/24; A23L 1/30; A23L 1/302; A61K 33/32; A61K 33/30; A23L 1/304

(52) U.S. Cl. 424/639; 424/641; 424/646; 424/655; 426/548; 426/72; 426/74

(57) ABSTRACT

A process is provided to improve the insulin sensitivity and glucose tolerance of an individual's metabolism. The process includes providing at a first location at least one polyol in an aqueous solution. A minor effective amount of a water soluble mineral composition comprising chromium picolinate is admixed with the aqueous solution to produce an aqueous polyol-chromium composition. The composition is dried and processed to produce solid chromium-infused polyol particles. The particles are packaged and transported to a second location. A selected quantity of the chromium-infused polyol particles are used to produce a food product selected from a group consisting of a liquid food including a liquid and at least fifty percent solids by weight, and a solid food composition. The food products produced with the mineral-infused polyol particles are packaged. The packaged food products are sold to the individual. The individual ingests the food product containing the mineral-infused polyol.

This application claims priority on U.S. Provisional Patent Application Ser. No. 60/844,004 filed Sep. 12, 2006.

This invention pertains to method and compositions to improve the metabolism of an individual.

Obesity and diabetes are epidemic in the United States and often cause a variety of illnesses, some of which are life threatening. One cause of these diseases is the poor dietary practices of many Americans, including, in particular, the intake of excessive amounts of sugar and fat, and including the failure to intake a sufficient quantity of recommended vitamins, minerals, and fiber.

Various studies have reported an association between diabetes mellitus (DM) and alterations in the metabolism of specific trace minerals. For example, impaired insulin release, insulin resistance and glucose intolerance in experimental animals and humans with DM have been linked to deficiencies of chromium, magnesium, selenium, vanadium, and zinc. Additionally the risk associated with these trace minerals is extremely low when used in moderation, therefore adding them to food ingredients is a relatively safe and effective means to aid the daily intake of these important micronutrients.

Chromium is a particularly desirable mineral because it has been shown to increase energy, improve weight control, and induce better carbohydrate metabolism. Insulin's ability to regulate glucose levels and lipid metabolism depends on its binding to specific receptors found in many peripheral tissues, eg., adipocytes, muscle, liver. Chromium increases the number of insulin receptors present in the target tissue; it also has shown to increase the binding of insulin to its receptors. Chromium has also shown to significantly reduce glycosylated hemoglobin (HbA_{1c}) which is key to maintaining long-term glucose control, which is correlated to the prevention of complications of diabetes, such as neuropathy and vascular disease.

Although the recommended daily dosage of chromium is relatively small, 120 mcg/day, the normal person's diet is far below this amount. Studies have shown that effective levels of elemental chromium range from 200 to 600 mcg. Chromium picolinate, chromium chloride, chromium nicotinate, polynicotinate, and niacin-bound chromium are highly bioavailable forms of chromium which may be added to food ingredients to increase consumers daily intake of this important micronutrient.

Deficiencies in magnesium have shown to be decremental to the enzyme activities of several metabolic pathways related to diabetes mellitus (DM). It is important in preventing the long-term complications of diabetes. The recommended daily allowance for magnesium is 320 mcg. Magnesium in the forms of oxide, gluconate, carbonate, chloride, citrate, gluconate and amino acid chelates are acceptable forms of this important micronutrient.

Selenium is an antioxidant which controls free radical production and helps prevent glucose intolerance and the complications related to Diabetes Mellitus. Effective ranges of this micronutrient range from 250 to 750 mcg per day. One available form of selenium is sodium selenate.

Vanadium, as sodium metavanadate or vanadyl sulfate, has shown an ability to mimic the actions of insulin. Additionally, human and animal studies have shown improved sensitivity to insulin. It has shown effectiveness at doses of 125 mg.

Decreased bioavailable zinc has shown to lead to an increase Diabetes Mellitus in some studies, since diabetics are more likely to have suboptimal status of zinc as a cofactor fro metalloenzymes that regulate the metabolism of carbohydrates, lipids, and proteins. The RDA of zinc in adults is 12-15 mg. Available forms of zinc are zinc acetate, and zinc gluconate

Biotin, also known as vitamin H or B7, has independently demonstrated its glucose and lipid-reducing effects as early as 1969; these effects were definitively shown in a double-blind 1980 study by M. W. Marshall, and others, that appeared in the journal, Artery. Biotin is important to healthy pancreatic and liver function. This is important because the pancreas' job is to produce the insulin that the body needs The liver's job is to act like the body's filter, taking things and converting them into substances the body can use or substances that can be easily disposed of. The RDA of Vitamin B7 is 300 mcg/day.

As a partial solution to the intake of excessive amounts of sugar and fat, many individuals consume low sugar, low calorie food formulations. Such food formulations often incorporate polyols and nutritional supplements. These food formulations are beneficial, but are not always homogenous or stable over time. Further, care must be taken when attempting to add minerals like selenium, chromium, zinc, magnesium and vanadium which are important in a person's daily diet but which are only required in small amounts. Food formulations containing these trace mineral compositions can tend to agglomerate in a food product or to coat particles in the food formulations so that the food formulation contains undesirably high localized concentrations.

We have discovered a new process to improve the metabolism, including insulin sensitivity and glucose tolerance, of an individual. The first step in the process is to provide at a first location at least one polyol in an aqueous solution. A minor effective amount of a water soluble mineral composition comprising chromium is admixed with the aqueous solution to produce an aqueous polyol-chromium composition. The composition is dried and processed to produce solid chromium-infused polyol particles. The particles are packaged and transported to a second location. A selected quantity of the chromium-infused polyol particles are used to produce a food product selected from a group consisting of a liquid food including a liquid and at least fifty percent solids by weight, and a solid food composition.

It is important to utilize the polyol particles in a solid food product or in a liquid food product containing at least fifty percent by weight solids because the water activity in such products is low, which minimizes the degree to which micronutrient infused polyol particles are dissolved and which, consequently, keeps in the particles chromium and other minerals or micronutrients that are infused in the polyol particles. This is preferred first because the minerals in infused polyol particles are generally dispersed evenly throughout the particles, and the particles are therefore relatively homogenous. It is also preferred, and important, because if the polyol particles dissolve, the infused minerals, vitamins, etc. are released into solution which can adversely affect the stability of the minerals, the homogeneity of the solution, the shelf life of the solution, and the safety of the solution in the event the minerals, vitamins, etc. agglomerate or combine in an undesirable fashion with other chemical components in the food product. Sorbitol or other polyols in liquid form often are at least 50% solids by weight.

The food products produced with the micronutrient-infused polyol particles are packaged. The packaged food products are sold to the individual. The individual ingests the food product containing the micronutrient-infused polyol.

Another embodiment of the invention comprises a food product to improve the carbohydrate metabolism of an individual. The food product comprises a bulk sweetener comprising solid micronutrient-infused food particles. Each of the infused food particles comprising a polyol and a minor effective amount of chromium infused in said particle. Each infused food particle can also include at least one composition selected from a group consisting of a water soluble selenium composition, a water soluble magnesium composition, a water soluble zinc composition, a water soluble vanadium composition, Vitamin B7, and fiber.

Each infused food particles can also be infused with at least one micronutrient from a group consisting of selenium, magnesium, zinc, vanadium, Vitamin B7 and fiber. The process of the invention can also be carried out utilizing an infused liquid polyol composition in place of the polyol particles noted above. The solid polyol particles are, however, presently preferred because of their ability in many solid or semi-solid foods to maintain minerals and other micronutrients homogeneously dispersed throughout the particles. The solid particles used in food preparations also are preferred as their stability or shelf-life is extended in a dry from rather than in a liquid form.

### Definitions

As used herein, a liquid food product is a composition that includes a liquid and at least fifty percent solids by weight. For example, a food composition consisting entirely of water is not a liquid food product. A milk shake ordinarily includes at least 50% by weight solids and is considered a liquid food product. Slurries like milk shakes, salsa, cake batters, pancake mix (after water or milk is added and the mix is ready to put on a griddle to make pancakes), etc. or semi-solid food products like custard, jello, etc. ordinarily will include at least 50% solids by weight and these compositions are, for the purposes herein, liquid food products.

As used herein, a solid food product is a food product that ordinarily will retain its shape when set on a surface without its packaging. Breads, cakes, crackers, frozen substantially rigid ice cream (popsicles, fudge bars, etc.), and macaroni (before it is cooked) are examples of solid food products.

As used herein, a solid bulk sweetener consists of mineral-infused polyol particles shipped in a container holding at least 55 pounds of the mineral-infused polyol particles.

As used herein, a liquid bulk sweetener is a mineral-infused liquid polyol composition shipped in a container holding at least 40 pounds of the mineral-infused liquid polyol composition.

As used herein, a polyol is a hydrogenated form of carbohydrate, whose carbonyl group (aldehye or ketone, reducing sugar) has been reduced to a primary or secondary hydroxyl group. Examples of polyols, both liquid and solid forms, are polygycitol, maltitol, isomalt, lactitol, sorbitol, mannitol, xylitol, erythritol, trehalose, tagatose, isomaltulose, sucromalt, glycerol, polydextrose, hydrogenated starch hydrosylate, arabitol, and maltotritol

As used herein, an infused polyol liquid or solid, is a polyol in which a mineral or other component or composition is generally homogenously dispersed through the polyol liquid or solid.

As used herein, a solid polyol particle contains at least 5 to 55 percent by weight of one or more polyols.

As used herein, a solid polyol particle infused with micronutrients contains at least 97%, preferably at least 99% by weight of one or more polyols.

As used herein, a polyol liquid including micronutrients therein contains at least 60 percent, preferably at least 70 percent, by weight polyol, contains less than 3% by weight, preferably less than 1% by weight, micronutrients, with the remainder of the polyol liquid consisting substantially of water.

As used herein, a polyol liquid contains at least 5 to 70 percent by weight of one or more polyols.

The micronutrients of primary importance herein comprise chromium, magnesium, zinc, selenium, vanadium, vitamin B7, and fiber. Fiber (or a vitamin or mineral) can be included in a solid polyol particle or in a polyol liquid in concentrations greater than 3%. When fiber (or a vitamin or mineral) is included in a solid polyol particle or in a polyol liquid in concentrations greater than 3%, the fiber is not considered a micronutrient. When fiber is includes in a solid polyol particle or in a polyol liquid in a concentration of 3% or less by weight, the fiber is considered to be a micronutrient.

The following examples are provided by way of explanation, and not limitation, of the invention.

### EXAMPLE 1

The following ingredients are provided.

| Ingredient (in liquid form) | Weight Percent |
|---|---|
| Sorbitol (HP-1) | 5 |
| Maltitol (HP-2) | 50 |
| Maltotritol (HP-3) | 15 |
| HP-11+ | _30 |
| Chromium picolinate | .0005 |

### The amount of water in the sorbitol is about 30% by weight; in the maltitol is about 15% by weight; in the maltotritol is about 20% by weight; and in the HP-11 is about 20% by weight.

The ingredients listed above are admixed to form a liquid polyol-chromium composition. The composition is packaged as a bulk sweetener and shipped to a desired location. At the desired location, a selected amount of the polyol-chromium composition is admixed with at least one other ingredient to form a food product. As used herein, a food product is a product that is ingestible by a human being or by an animal and that includes fats, proteins, mineral, vitamins and/or carbohydrates.

### RDI and the Amount of Mineral in a Polyol

As noted, minerals (or other micronutrients) other than chromium can be infused in a polyol in accordance with the invention. The concentration of a mineral in a polyol depends on the RDI (Recommended Daily Intake), levels disclosed in previous research and/or on the amount of the polyol desired in the final food product, as desired. For example, if a serving of a food product is one hundred and fifty grams, if the mineral infused polyol of the invention, comprises 10% by weight (i.e., fifteen grams) of the food product, if the mineral infused in the polyol is chromium, and if the goal is to include in the serving one third (i.e., 40 mcg) of the 120 mcg RDI of chromium, then in order to include this amount of chromium, the mineral-infused polyol liquid mineral-infused polyol would include about 0.00026% by weight of chromium picolinate; and, typically, the amount of chromium picolinate in a liquid mineral-infused polyol would be in the range of 0.01 to 0.00001% by weight. Since the liquid mineral-infused polyol is roughly about 25% by weight water, the amount of chromium picolinate in dry mineral infused polyol particles would be in the range of about 0.0125% to 0.0000125% by weight. Selenium has a RDI of 85 mcg and would like, chromium, comprise 0.01 to 0.00001% by weight of a mineral infused liquid polyol and would comprise 0.0125% to 0.0000125% by 9′ weight of dry mineral infused polyol particles. Magnesium has a RDI of 320 mg, and in a liquid mineral-infused polyol magnesium would comprise about 0.01% to 10% by weight of the liquid polyol, and would comprise 0.0125% to 16.25% by weight of dry mineral infused polyol particles. There is no RDI for vanadium but studies have shown positive effects at levels of 125 mg for sodium metavanadate, and in a liquid mineral-infused polyol vanadium would comprise 0.0001% to 10.2% by weight, and in dry mineral-infused polyol particles would comprise 0.00125% to 20.5% by weight of the particles. The recommended RDI for Vitamin B7 is 300 mcg/day and in a liquid mineral-infused polyol Vitamin B7 would comprise 0.001% to 20% by weight, and in dry mineral-infused polyol particles would comprise 0.0125% to 16.5% by weight of the particles. The RDI for zinc is 12 mg, and in a liquid mineral-infused polyol zinc would comprise 0.001% to 1.2% by weight, and in dry mineral-infused polyol particles would comprise 0.00125% to 1.5% by weight of the particles. As would be appreciated by those of skill in the art, the concentration of a particular mineral or other component or composition that is infused polyol can vary as desired to achieve the desired concentration in the finished food product serving size.

### Water Soluble Mineral Compositions

The mineral composition (or other composition that includes desired components other than minerals) that is utilized to infuse a mineral into a polyol in accordance with the invention is at least partially soluble in water. Consequently, the composition utilized to provide chromium in a liquid polyol (where liquid polyols comprise aqueous solutions or other aqueous mixtures) is typically chromium picolinate; the compositions used to provide selenium in a liquid polyol typically comprise selenomethionine or cystine; the compositions used to provide magnesium in a liquid polyol typically comprise magnesium citrate, magnesium gluconate, or magnesium oxide; the compositions used to provide vanadium in a liquid polyol typically comprise sodium metavanadate or vadyl sulfate; the composition used to provide Vitamin B7 in a liquid polyol typically comprise Biotin; and, the composition typically used to provide zinc in a liquid polyol typically comprises zinc acetate.

### Fiber

As noted, fiber can be utilized in mineral-infused polyols produced in accordance with the invention. The RDI for fiber is 38 g/day. Examples of fiber include agave fiber, chicory, or beet fiber. The weight percent of fiber in a liquid polyol or in a solid polyol is in the range of 1% by weight to 95% by weight, preferably 35% by weight to 75% by weight. Sources for liquid versions of fiber may include agave syrup with soluble insulin.

### EXAMPLE 2

The liquid polyol-chromium composition of Example 1 is further processed as follows to produce solid mineral-infused polyol particles.

First, the liquid polyol-chromium composition is heated to 330 degrees F. and is then subjected to a vacuum of 24 to 27 inches of mercury while under agitation. Further additives and ingredients (such as selenium and zinc water soluble mineral compositions) can, if desired, be added at this point. The 330 degree F. temperature and the vacuum are maintained until the residual moisture in the composition is less than 0.5% by weight. Use of a vacuum facilitates removal of moisture from the composition.

After the residual moisture in the composition is less than 0.5% by weight, the composition is aerated to improve appearance and fracturing during milling. The composition is then formed into a rope by extruding the composition. The “rope” is stamped to form three-quarters of an inch by three-quarters of an inch pillows. If desired, further additives and ingredient can be added to the composition before, during, or just after the extrusion process.

The pillows are allowed to cool to room temperature and cure. After the pillows cool and cure, they are classified using a Fitzmill or other fractioning classifier to produce particles of chromium-infused polyol. Further additives and ingredients can be added to the product during the fractioning/classifying process. The resulting particles of chromium (or other mineral(s))-infused polyol can be used as is or can be combined with other ingredient to make other finished products.

The amount of air utilized during the aeration portion of the process noted above can be varied in order to alter the product bulk density as desired. Altering the bulk density is one way to vary and control the particle size of the resulting mineral infused-polyol particles.

Nitrogen can be utilized during any desired stage of the above described process either as a cooling agent or to displace ambient air.

A liquid or solid mineral infused polyol produced in accordance with the invention can comprise one or more polyols. In one preferred embodiment of the invention, dry mineral-infused polyol particles having the following polyol concentrations:

| Polyol | Weight Percent |
|---|---|
| Sorbitol (HP-1) | 0.1 to 15 (dry basis) |
| Maltitol (HP-2) | 0.1 to 85 (dry basis) |
| Maltotriitol (HP-3) | 10 to 25 (dry basis) |
| HP-11+ | 0 to 60 (dry basis) |
| Water | 0 to 5 |

By way of example, and not limitation:
Sorbitol may comprise one or more of the following: SORBOGEM™ and SORBO™ by SPI Polyols; NEOSORB™ by Roquette; SORBITOL SOLUTION and CRYSTALLINE SORBITOL by Archer Daniels Midland (ADM); and SORBODEX™ by Merck & Co., Inc.
Maltitol may comprise one or more of the following: MALTISWEET™, STABILITE™, and HYSTAR™ by SPI Polyols; MALTISORB™ and LYCASIN™ by Roquette; MALTIDEX™ and MALBIT™ by Cerestar; and, LESYS-T™ and AMALTY™ MR by Towa Chemical Industry Co., Ltd.
Maltotriitol may comprise one or more of the following: MALTISWEET™, STABILITE™, and HYSTAR™ by SPI Polyols; LYCASIN™ by Roquette; and MALTIDEX™ by Cerestar.
HP-11+ may comprise one or more of the following: MALTISWEET™, STABILITE™, and HYSTAR™ by SPI Polyols; LYCASIN™ and NUTRIOSE™ by Roquette; MALTIDEX™ by Cerestar; FIBERSOL™ by Matsutani Chemical Industry Co.; and, POLYDEXTROSE™ by Danisco.
   The water may be de-ionized water, USP water, de-chlorinated water, mineral water, treated water, or tap water. The preferred water is deionized water.

The finished product (per the process set forth in Example 2) is a solid, free-flowing particle with moderate sweetness; is fully soluble; and, is fully dispersable. The glass transition temperature is between 25 to 75 degrees C. with the preferred embodiment having a transition temperature between 45 and 55 degrees C. The average molecular weight (AMW) is between 344 and 1800 grams/mole with the preferred embodiments having an AMW between 348 and 600 grams/mole. The surface area is between 0.01 and 0.8 square meters/gram with the preferred embodiments having a surface area between 0.15 and 0.4 square meters/gram. The aerated bulk density is between 0.25 and 0.8 g/cubic centimeter, with the preferred embodiments having an aerated bulk density between 0.45 and 0.65 g/cubic centimeter. The molten viscosity at 115 degrees C. is between 600,000 and 1,200,000 cps with the preferred embodiments having a molten viscosity at 115 degrees C. between 850,000 and 950,000 cps. The liquid/molten state solids range is between 97-100%, with the preferred embodiments having a range of 98.5% to 99.5%. Additional parameters which can, if desired, be evaluated are relative sweetness, caloric value, pH, water activity, compressibility, tap density, particle size, angle of repose, specific heat capacity, conductivity, color, and heat of solution.

### EXAMPLE 3

The following ingredients are provided.

| Ingredient | Weight (Grams) | |
|---|---|---|
| Ingredient | % | grams |

For Step #1

| | | |
|---|---|---|
| Cake Flour | 24.13 | 1000 |
| Granulated Sugar | 14.5 | 600 |
| Mineral Infused Polyol produced in Example 2 above | 14.5 | 600 |
| Whole Egg Solids | 2.41 | 100 |
| Nonfat Milk Solids | 1.8 | 75 |
| Salt | 0.72 | 30 |
| Baking Powder | 0.15 | 62.5 |

For Step #2

| | | |
|---|---|---|
| Water, 75 degrees F. | 15 | 625 |
| Flavoring, BLV (C.F. # 2182) | 0.12 | 5 |
| Liquid Egg Color | 0.06 | 2.5 |

For Step #3

| | | |
|---|---|---|
| Shortening | 6 | 250 |
| Cake emulsifier | 0.48 | 20 |

For Step #4

| | | |
|---|---|---|
| Water, 75 degrees F. | 7.2 | 300 |

For Step #5

| | | |
|---|---|---|
| Water, 75 degrees F. | 11.5 | 475 |

The mixing procedure is as follows:

### In Step #1: Use A-200 mixer with twenty quart bowl and a paddle beater agitator to dry blend in the ingredients noted above for use in Step #1. Mix for one minute at low speed to produce blended dry mix composition.

### In Step #2: Add to the blended dry mix composition of Step #1 the ingredients noted above for use in Step #2. Stir at low speed to produce flavored blended composition.

### In Step #3: Add to the flavored blended composition of Step #2 the ingredients noted above for use in Step #3. Mix for one minute at low speed and for three minutes at medium speed to produce shortening blended composition.

### In Step #4: Add to the shortening blended composition the water noted above for use in Step #4. Mix for one minute at low speed, and then for two minutes at medium speed to produce thinned blended composition.

### In Step #5: Add to the thinned blended composition of Step #4 the water noted above for use in Step #5. Mix for two minutes at slow speed to produce final cake batter.

Use 400 gm of the final cake batter of Step #5 in each eight inch layer cake pan and bake for twenty-three to twenty-seven minutes at 360 degrees F.

### EXAMPLE 4

Stir together:
One pound brown sugar
Eight ounces of mineral-infused polyol liquid produced in Example 1 above.
One ounce salt
Six ounces flour
One ounce cinnamon
One-fourth ounce ginger
One-half ounce vanilla

Add and mix well:
One #10 can pumpkin
One pound eight ounces honey
One pound fourteen ounces water
Four ounces melted butter

Add and mix in:
Four pounds of water
Two pounds whole eggs, break well before adding

Let the mix set for about one hour before using. Then stir well and pour into shells in the oven, stirring occasionally while filling the shells. Bake at 425 degrees F. for fifteen minutes; reduce temperature to moderate (350 degrees F.) and continue baking 25 to 30 minutes, or until a knife inserted in center comes out clean.

### EXAMPLE 5

A liquid composition is produced to coat chewing gum. The composition includes 83 to 97% by weight of sorbital polyol in liquid form. The liquid sorbital polyol is infused with chromium in the form of chromium picolinate. The liquid sorbitol polyol is infused with chromium in the manner described in Example 1 above.

### EXAMPLE 6

A reduced sugar, low calorie ice cream is produced using a liquid maltitol polyol infused with chromium in the form of chromium picolinate. The liquid maltitol polyol is infused with chromium in the manner described in Example 1 above. The mineral-infused polyol is utilized in place of traditional sweeteners like Maltisweet™, produced by SPI.

### EXAMPLE 7

A control group of 100 individuals is selected. Fifty of the individuals in the control group have Type 1 diabetes. The remaining fifty of the individuals in the control group have Type 2 diabetes.

A second test group of 100 individuals is selected. Fifty of the individuals in the test group have Type 1 diabetes. The remaining fifty of the individuals in the test group have Type 2 diabetes.

Each of the individuals in the control group and the test is overweight because each of the individuals has a greater than desired proportion of body fat. Some of the individuals are significantly overweight, others are only slightly overweight. The proportion of significantly overweight individuals (more than 50 pounds overweight) in the control group is equivalent to the proportion of significantly overweight individuals in the test group. The proportion of slightly overweight individuals (10 pounds or less overweight) in the control group is equivalent to the proportion of slightly overweight individuals in the test group. The proportion of moderately overweight individuals (11 pounds to forty-nine pounds overweight) in the control group is equivalent to the proportion of moderately overweight individuals in the test group. Each individual in the control group and in the test group takes a supplemental dose of insulin each day.

All of the individuals in the control group and in the test group each eat for an initial two week period a prescribed low sugar diet (comparable to a diet offered by Weight Watchers) in which simple sugars (granular sugar, frostings, syrups, hot fudge, etc.) not eaten and in which certain complex carbohydrates (breads, corn, peas, pasta, etc.) are limited. The diet is a relatively balanced diet and includes portions of fruits, vegetable, proteins, grains, etc. At the end of the two week period, the insulin sensitivity of each member of each group is determined. The insulin sensitivity of each individuals having Type 1 diabetes is determined by first filling in the blanks below:

$$\begin{matrix}Your weight ( lbs . ) / 4 = \_\_\_ \_ units . & Line 1 \\ Your total daily insulin dose ( all insulins ) = units . & Line 2\end{matrix}$$

The answer from line 1 (the individual's weight divided by 4) gives the individual's estimated need for insulin. If the actual insulin dose on line 2 is close to this number and is effective in controlling the metabolism of sugar, then the individual has a normal sensitivity to insulin. If the value on line 2 is less than line 1 (and the insulin dosage is effective in controlling the metabolism of sugar), then the individual has excellent insulin sensitivity. If the value on line 2 is much greater than the answer from line 1, then the individual's insulin sensitivity may be lower or the individual may be on too much insulin. Determining the sensitivity to insulin for an individual with Type II diabetes is more complicated because each individual varies in how much insulin their own pancreas produces and in how resistant they are to insulin, but procedures for determining the insulin sensitivity of individuals with Type II diabetes are well known and will not be described herein.

The average daily amount of chromium, selenium, magnesium, zinc, vanadium, and vitamin B7 in the low sugar diet eaten by each member of the control and test groups during the initial two week period is determined to be about 10 mcg, 7 mcg, 40 mg, 2 mg, and 30 mg (of sodium metavanadate), respectively.

Immediately following the initial two week period during which the low sugar diet is eaten by each member of the control and test groups, the low sugar diet is eaten by each member of the control and test groups for an additional two week period. However, the test group also ingests each day one or more food products that include mineral-infused polyols that supply 120 mcg per day of chromium. Foods containing the mineral-infused polyols are consumed at the same meal with foods containing complex carbohydrates. The control group continues to eat the low sugar diet which provides an average of 10 mcg of chromium per day.

After the additional two week period is over, the insulin sensitivity of the control group and of the test group are again evaluated. The insulin sensitivity of the members of the control group is generally unchanged. The insulin sensitivity of the members of the test group is improved.

### EXAMPLE 8

The process of EXAMPLE 7 is repeated, except that the mineral-infused polyols supply 85 mcg of selenium a day instead of supplying chromium. Similar results are achieved.

### EXAMPLE 9

The process of EXAMPLE 7 is repeated, except that the mineral-infused polyols supply 320 mcg of magnesium a day instead of supplying chromium. Similar results are achieved.

### EXAMPLE 10

The process of EXAMPLE 7 is repeated, except that the mineral infused polyols supply 12 mg of zinc a day instead of supplying chromium. Similar results are achieved.

### EXAMPLE 11

The process of EXAMPLE 7 is repeated, except that the mineral-infused polyols are replaced by micronutrient infused polyols that provide 300 mcg/day of Vitamin B7. Similar results are achieved.

### EXAMPLE 12

The process of EXAMPLE 7 is repeated, except that the mineral-infused polyols supply 125 mg per day of sodium metavanadate. Similar results are obtained.

### EXAMPLE 13

The process of EXAMPLE 7 is repeated, except that the foods containing the mineral-infused polyols are not ingested at the same meals as food containing complex carbohydrates, but the foods containing the mineral-infused polyols are consumed at least several times throughout each day, once in the morning, once in the afternoon, and once in the evening. Similar results are obtained.

Batch vs. Continuous Process

The above described process can be carried out either with a batch process or a continuous process or a combination of the two. The order of addition of the ingredient can be varied to yield acceptable product depending on the types of ingredients being blended together and their respective physical characteristics.

### Equipment

Examples of equipment and processes than can be utilized during the above production process include drum crystallization, spray dry, extrusion, fluid bed dryer, dryblend/agglomeration, and block crystallization.

### Additional Ingredients

Additional additives and/or specialty ingredients can be added during the processes described above to yield a mineral-infused polyol with further product features and benefits. For example, during the production of mineral-infused polyol the following can be added:
Low digestible carbohydrates (LDCs) such as sugars, polyols, and soluble and insoluble fiber.
High digestible carbohydrates (HDVs) such as sugars, cereal syrups, maltodextrins, and starches.

In other embodiments of the invention, mineral-infused polyol (in liquid or solid form) is admixed with other ingredients via any desired process:
Mineral-infused polyol is admixed with extrusion crystallized LDCs (i.e., maltitol, isomalt, lactitol, sorbitol, mannitol, cylitol, erythritol, trehalose, tagatose, kalinose, temulose, etc.)
Mineral-infused polyol is admixed with solution crystallized LDCs.

Mineral-infused polyol is admixed with block crystallized LDCs.

One product that can result from the process variations described above is a solid polyglycitol (HSH, including maltitol syrup) or LDC (maltitol, isomalt, lcatitol, sorbitol, mannitol, xylitol, erythritol, trehalose, tagtose, osmaltulose, sucromalt) composition with free-flowing characteristics and shelf stability. Additionally, the high molecular weight portion can include other LDC compositions such as fiber (including, but not limited to, insulin, FOS, polydextrose, resistant maltodextrins, arabinogalactans, gums, etc.). Corn syrups can be combined with the various mineral-infused polyols as well.

Enzymes can be utilized in conjunction with the mineral-infused polyol of the invention. For example, an enzyme for the hydrolysis of gluten can be included in polyols utilized in the bakery industry. An enzyme to convert lactose or to reduce allergenic properties can be utilized in polyols utilized in the dairy industry. An enzyme to increase the texture of mechanically processed fruit and vegetable can be utilized in polyols utilized in connection with processing such fruits and vegetables.

### Specialty Ingredients

Specialty ingredients such as flavors, colorants, and sweeteners can be incorporated in the mineral-infused polyol of the invention to adjust the organoleptic properties of the finished product. For example, a chewing gum coating formulation can use a mineral-infused polyol complexed with titanium dioxide in order to produce a finished coated product with a more opaque, white appearance, while the dragee formula incorporates a second mineral-infused polyol that includes sucralose to give the gum center a greater sweetening profile.

### Additional Ingredients

Some examples of other compositions that can be integrated in or used with the mineral-infused polyols of the invention are:
Fiber (insoluble and soluble)
Calcium
Tooth whiteners
Electrolytes
Digestive aids
Cholesterol inhibitors (for example, flavonols)
Osmotic pressure regulators
Folic acid
Nutritional supplements
OTC drugs
Homeopathic remedies

### Advantages of Mineral-Infused Polyol

One advantage of the mineral-infused polyol LDC composition of the invention is that is can be used to decrease sugar and calories and to increase the available fiber in foods.

In the case of the addition of fiber, another advantage of the mineral-infused polyol LDC composition of the invention is that it can be used to inhibit the intake of cholesterol in the small intestine and to therefore decrease the intake of harmful sterols into the blood stream.

A further advantage of the mineral-infused polyol composition of the invention is that when the polyol composition includes a fiber like insulin, it can increase the solubility or dispersability of the fiber in finished food products like, for example, baked goods.

### Applications in Food Industry

The micronutrient-infused polyols of the invention can be utilized in any desired food product. Some compositions in the food industry in which the micronutrient-infused polyols of the invention can be utilized include:
Syrups (dries vs. powder blends)
Confections (chocolate, caramels, fondants, creams, nutritional bars, hard candy, chewing gum, etc.)
Sweeteners (high intensity and natural strength)
Flavorings (natural, artificial, powder, liquid, oil-based, aqueous, etc.)
Colorants (dyes, pigments, lakes, etc.)
Baked goods (cookies, cakes, dry mixes, cremes, etc.)
Dry mixes
Dairy (ice creams, dry mixes, specialty items, etc.)
Frozen desserts
Bulking agents
Viscosity modifiers
   The micronutrient-infused polyols of the invention can also stand alone and function as on their own as a food product. For example, micronutrient-infused particles can be added to cereal or other food products when such products are consumed, or can be consumed at some time before or after the consumption of such products. It is, however, preferred in the practice of the invention that micronutrient-infused polyols of the invention be consumed simultaneously with or immediately before or after (i.e., within thirty minutes before or after) a food product so that the micronutrients which assist an individual's metabolism are available to the individual's digestive system at the time the food product is consumed, particularly if the food product contains a simple sugar or complex carbohydrate, or, if the food product is eaten with another food product that contains a simple sugar or complex carbohydrate. Alternatively, if the micronutrient-infused polyols are not ingested at the same time as food products containing simple sugars or complex carbohydrates, the micronutrient-infused polyols should be ingested by an individual at least two spaced apart times, preferably at least three spaced apart times, during a day to try to insure that the micronutrients are available in the individual's body throughout the day.

It is preferred that when a food containing simple sugars or complex carbohydrates is produced, the food be manufactured with and include liquid or solid micronutrient-infused polyols manufactured in accordance with the invention. This insures that the micronutrient-infused polyol will be consumed at the same time as the simple sugar or complex carbohydrate and that the micronutrient in the polyol will be available to assist in the metabolism of the simple sugar or complex carbohydrate or other targeted composition in the food product.

Having described my invention in such terms as to enable those of skill in the art to make and practice it, and having described the presently preferred embodiments thereof, I Claim:

1. A method to improve carbohydrate metabolism in an individual, comprising the steps of
(a) providing at a first location least one polyol in an aqueous solution;
(b) admixing a minor effective amount of a water soluble mineral composition including chromium to said aqueous solution to produce an aqueous polyol-chromium composition;
(c) drying and processing said polyol-chromium composition to produce solid chromium-infused polyol particles;
(d) packaging said chromium-infused polyol particles;
(e) transporting said package chromium-infused polyol particles to a second location;
(f) utilizing a selected quantity of said chromium-infused polyol particles at said second location to produce a food product selected from a group consisting of a liquid food including a liquid and at least fifty percent solids by weight, and a solid food composition;
(g) packaging said food product;
(h) selling said packaged food product to the individual; and,
(i) said individual ingesting said food product.

2. A food product to improve the carbohydrate metabolism of an individual, comprising a bulk sweetener comprising solid infused food particle, each of said infused food particles comprising a polyol and a minor effective amount of chromium infused in said particle.

3. The method of claim 1 wherein in step (b) said mineral composition also includes at least one composition selected from a group consisting of a water soluble selenium composition, a water soluble magnesium composition, a water soluble zinc composition, and a water soluble vanadium composition, Vitamin B7, and fiber.

4. The food product of claim 2 wherein said infused food particles are each also infused with at least one nutrient from a group consisting of selenium, magnesium, zinc, vanadium, Vitamin B7 and fiber.

5. A method to improve insulin sensitivity and glucose tolerance in an individual, comprising the steps of
(a) providing at a first location least one polyol in an aqueous solution;
(b) providing a water soluble mineral composition including chromium,
(c) admixing a minor effective amount of said water soluble mineral composition to said aqueous solution and producing an aqueous polyol-chromium composition;
(d) packaging said polyol-chromium composition;
(e) transporting said package chromium-infused polyol composition to a second location;
(f) utilizing a selected quantity of said chromium-infused polyol composition at said second location to produce a food product selected from a group consisting of a liquid food including a liquid and at least fifty percent solids by weight, and a solid food composition;
(g) packaging said food product;
(h) selling said packaged food product to the individual; and,
(i) said individual ingesting said food product.
